(19) United States
(12) Patent Application Publication
(10) Pub. No.: US 2007/0160977 A1
(43) Pub. Date: Jul. 12, 2007

(54) METHOD FOR DETECTING MOLECULES OR CHEMICAL REACTIONS BY DETERMINING VARIATION OF CONDUCTANCE

(76) Inventors: Linda Olofsson, Onsala (SE); Sven Enerback, Mölndal (SE)

Correspondence Address: GAUTHIER & CONNORS, LLP, 225 FRANKLIN STREET, SUITE 2300, BOSTON, MA, 02110, US

(21) Appl. No.: 11/684,212

(22) Filed: Mar. 9, 2007

Related U.S. Application Data
(63) Continuation of application No. 11/318,717, filed on Dec. 27, 2005.
Application No. 11/318,717 is a continuation of application No. 10/437,761, filed on May 14, 2003, now abandoned.
Application No. 10/437,761 is a continuation of application No. PCT/SE01/02616, filed on Nov. 26, 2001.

(30) Foreign Application Priority Data
Nov. 24, 2000 (SE) 0004334-9

(51) Int. Cl. C12Q 1/00; G01N 17/04

(52) U.S. Cl. 435/4; 205/777

(57) ABSTRACT

The present invention relates to a detection method for detecting molecules and/or chemical reactions, whereby target molecules are attached to a series of electrodes, that the series of electrodes with molecules are subjected to assay said molecules or other molecules, whereupon the variation of conductance is determined.

### TECHNICAL FIELD

The present invention relates to molecular and electron spectroscopy, in particular electron spectroscopy of biological molecules.

### BACKGROUND OF THE INVENTION

In the quantitative and qualitative analysis of the presence or absence of different biomolecules different assays are used, such as ELISA, reporter groups using fluorescent groups for providing of information of events, and others. Most systems includes a number of steps to be carried out to provide the necessary information.

The object of the present invention is to reduce the number of steps needed to carry out an assay with regard to quantitative and qualitative analysis of biomolecules.

U.S. Pat. No. 5,827,482 relates to a molecular detection apparatus having a first gate, a first molecular receptor proximate to the first gate, a second transistor having a second gate, and second molecular receptor proximate to the second gate, whereby a differential voltage is applied between the first and second gates to enhance binding difference between the first molecular receptor and the second molecular receptor.

### DESCRIPTION OF THE PRESENT INVENTION

It has now surprisingly been shown possible to solve this problem by means of the present invention which is characterized in that target molecules are attached to a series of electrodes, that the array is subjected to assay molecules, whereupon the variation of conductance and/or impedance is determined.

The present invention makes it possible to detect, with a sensitivity of down to one molecule, molecule A in small volumes (μl). The molecule B, to which A binds specifically, covers, partially or completely, a series of electrodes on a chip. The chip will be exposed to a solution, the contents of A of which one wants to determine whereupon the binding between A and B molecules is detected by means of one out of four detection principles that are available according to the present invention, viz:
Impedance determination—at the binding-in the dielectricity constant between the electrodes is changed whereby the capacitance is changed. The resistance may, under certain circumstances be changed as well, as the molecule can be more or less conducting/isolating.
Tunnelling—a binding would change the tunnel barrier and so the tunnel characteristics of the junction.
SET—single electron tunnelling transistor—is an ultra sensitive charge measurement device. Charge changes as small as a thousandth of an electron charge can be determined. The molecules can be a part of the two tunnel barriers, which SET consists of. Then the detection consists of a combination of a changed tunnel characteristics and change of charge.
SET—single electron tunnelling—in the neighbourhood of a reaction will detect the change of charge when the reaction takes place.

The detection principle is measurement of conductance variations, which can be detected by AC or DC measurement techniques. The electrodes used for these measurements are functionalised by for example self-assembly of molecules for recognition or binding of the target molecules. The dimension of the electrodes is made such that the conductance could be affected by very low number of molecules, i.e., down to molecular dimensions. The DC technique measure the electron tunnelling rate in the adsorbed molecules and can detect variations induced by structural changes, chemical reactions or adsorption of other molecules. E.g., the electron tunnelling rate in a DNA molecule can be measured and the adsorption of a protein along the DNA-strand, could be detected as a variation of the tunnelling characteristics. The AC conductance can be used for the detection of changes in the dielectric properties of the medium between the electrodes. When a molecule is adsorbed, the permittivity change which can be detected by measuring the impedance (i.e., capacitance) of the junction between the electrodes. The adsorption of specific target molecules in the region between the electrodes could be accomplished by for example functionalising the surface by self-assembly.

As an example one can mention carboxylic acids on oxide bearing metals, such as silver, aluminium, and titanium, chloro- and alkoxy-silanes which could be deposited on most substrates under proper conditions and organo sulphur molecules on noble metals, such as gold, platinum, palladium.

Impedance is used at 0 kHz to 8 GHz, preferably at 20 to 1000 kHz, whereby some type of frequency adaptation of wires and joints has to be made to avoid background noise and disturbances. Normally the impedance is measured at room temperature up to 100° C. as at higher temperatures thermal noise occurs.

The invention further allows a set-up of arrays of electrodes to detect and determine a spectrum of molecules.

In accordance with a preferred embodiment the detection is determined by means of impedance spectroscopy.

In accordance with a further preferred embodiment the detection is determined by means of capacitance spectroscopy.

In accordance with a still further preferred embodiment the detection is determined by means of tunnel spectroscopy.

In accordance with another preferred embodiment the detection is determined by means of single electron tunnelling spectroscopy, wherein preferably the detection is determined by means of single electron tunnelling spectroscopy arranged in the vicinity of the reaction and arranged to detect the exchange of charge.

In accordance with a preferred embodiment the molecules are organic chemical molecules.

In accordance with another preferred embodiment the molecules are biomolecules.

In accordance with a further preferred embodiment the molecules are inorganic chemical molecules.

In accordance with a preferred embodiment the molecules to be detected are attached to a substrate having no conductive top layer, wherein preferably the top layer is of silicon, or more preferably of glass.

On a chip the different electrodes can be covered by different molecules B (B1, B2, B3 . . . etc.) which each individually detects a specific molecule A, which in turn causes that it should be possible to use one single chip to analyse e.g., a whole blood sample. The chip is then mounted on a carrier, which can be connected directly to a computer and thus the result can be read directly, and actually in real time, on the computer screen.

The invention can be applied within medicine for analysing a blood sample, DNA, sequence determination, protein analyses, environmental care for detecting small amounts of pollutions in lakes etc., exhaust purification for controlling the efficiency of such, air pollutants for controlling the contents of contaminants, allergens etc., food industry for detecting toxic or non-inert contaminants in food. As a conclusion it can be stated that the invention can be used wherever small amount so one or more molecules need to be detected.

In a preferred embodiment of the invention micro to nano-structures on a chip will enhance the signal obtained in the examples given above.

In a further preferred embodiment the electrodes are present as elevated dots on a chip onto which the substances to be measured are applied, whereupon an electric field is applied, the changes of which is then recorded.

It should be noted that tunnelling includes nanodistances while capacitance measurement includes nano- to micrometer distances.

For SETs metals, metal oxides. SiO₂, SiN₃ are typically used in the fabrication of the device. The structures need to be small in order to function at room temperature. If the structures are larger than 10 nm cooling of the device is needed in order to function. Cooling can be achieved by liquid helium, liquid nitrogen or by using a cryostat.

The substrates should have an insulating layer on top, for example 1 mikrometer SIO on a top of a silicon wafer. For the AC measurements it is essential that the substrate does not absorb too much of the field applied and therefore the substrate should be chosen such that total dielectric constant of the substrate is much lower than the dielectric constant of the system studied, for example a thick glass substrate when measuring in water systems.

Further, single molecule adsorption is possible to detect by ultra-sensitive electrometers, such as single electron tunnelling transistor (SET). In this case variations of the electrical environment induced by the presence of biomolecules or chemical reactions in the vicinity of the transistor can be detected. The single electron transistor can for example be made of small metallic particles with nanometer dimensions. SET has an ability to detect charges which corresponds to only a fraction of the electron.

Manufacture of these devices for these detection methods could be done using standard lithographic techniques such as photolithography and electron beam lithography combined with self-assembly and chemical synthesis of nanoscale objects as described in references 1 and 2.

Capacitance Spectroscopy

Different biomolecules have dielectric constants. Adsorption of biomolecules in a gap between two electrodes can thus be detected as variations of the capacitance. Capacitance is easily monitored by AC measurement techniques.

For a parallel-plate capacitor, cf. FIG. 1 the capacitance is given by
C=εᵣ^{*}ε_{O}A/a,
where ε_{O}^{*}εᵣ is the permittivity, ε_{O} is the dielectric constant, a is the height and A is the area.

Charging and discharging of a capacitor follows from FIG. 2, wherein if an AC-voltage, V, is applied the capacitance can be determined from
V=I*(R-i/ω*C),
wherein ω/2TT is the frequency of the AC-voltage. A lock-in amplifier can for example be used to measure this.
Tunnelling Spectroscopy

Electrons can tunnel through thin insulating barriers [3], such as different oxides and polymers. The tunnelling effect is used in for example, the scanning tunnelling microscope where a metallic tip is scanned over a conducting surface and the tunnelling current is measured and used to regulate the distance between tip and surface. The present invention makes use of variations of tunnelling current to detect changes of molecules, which are placed between two electrodes. The tunnelling current is strongly dependent on the distance between the electrodes, but also on the tunnel barrier. A molecule, such as DNA or other biomolecule can be assembled between the two electrodes and act as a tunnel barrier for electrons. A change of the tunnel characteristics can be induced by structural or chemical alterations. Changes in molecular structure, for example, an opening in a double stranded DNA, which forms two single stranded branches will change the tunnelling characteristics. Also an adsorption of another molecule on the first one will alter the probability of tunnelling between the electrodes. Tunnelling spectroscopy can hence be used for detection of small quantities of molecules. The electrodes for such spectroscopy can be made in large numbers on a chip where different electrodes can be modified by self assembly of molecules with high affinity to a target.

A complete understanding of electron tunnelling through molecules such as DNA does not exist today but intense efforts are made world-wide to establish theoretical model electron transport in both organic molecules and biomolecules. Some recent experiments have shown semi-conducting electron transport in DNA molecules [4, 5].

Single Electron Tunnelling Transistor

The single electron tunnelling transistor, SET, is a very sensitive electrometer, which can detect charge variations much smaller than the electron charge [6, 7, 8]. A sensitive electrometer can be used to detect electron transfer reactions or adsorption of charged objects in the vicinity of the transistor. The most common SET are operating below 1K, but during the last couple of years, several research groups have reported room temperature operation. The crucial point for high temperature operation of these devices is the dimension of a small conducting island. Dimensions as small as 10 nm and less are required for enabling room temperature operation. The present invention uses the ultra-sensitive SET for detection of molecules and molecular charge transfer reactions in the vicinity of the SET as well as in the SET as such. SET is working at 10 nm or less normally at room temperature, herein 20° C., but can be used in the range of 0 to 100° C. when it comes to biomolecules.

A schematic picture of a SET transistor is given in FIG. 3. The SET is an extremely charge sensitive device, which consists of a conducting island separated from the source and drain leads by two tunnel junctions. A gate is capacitively coupled to this structure by which the charge distribution of the island is changed. This results in a periodic modulation of the voltage across the SET (alternatively the current through the SET).

When a voltage is applied across the double junction the junction capacitance will be charged. Electrons will not tunnel through the barriers until the voltage across the juntion corresponds to a charging energy of a single electron, E_{c}=e²/2C, i.e., V=e/2C, where C is the total capacitance of the junctions. To understand this we must look at how the charging energy of a capacitance depends on the charge: E=q²/2C. This parabolic curve is shown in FIG. 4. From this figure it can be concluded that if the charge on the capacitance is smaller than e/2, the energy would increase if an electron would tunnel. This region of no current is called Coulomb blockade. If the charge on the capacitance is larger than e/2 the energy would decrease if an electron would tunnel and therefore tunnelling will occur. The charge on the capacitance can be tuned by applying a voltage on the gate.

After a tunnel event, the potential of the island increases and prevents other electrons from tunnelling and then the next electron cannot tunnel until a half electron charge is accumulated on the junction capacitor. Hence, the electrons tunnel one by one. The potential of the metal island between two tunnel barriers can be controlled by an external electric field. By applying a voltage to the gate, the current through the SET can thus be modulated. As the voltage of the gate is changed there will be a suppression of the Coulomb blockade, i.e., the width of the Coulomb blockade is varied between its maximum and zero volt, which latter means total suppression. The modulation is periodic with each period corresponding to one electron charge in the single electron tunnelling transistor, SET. This is why the SET is such a charge sensitive device, viz. only a fraction of the electron charge difference on the gate gives a large difference in tunnel current. It can thus be used to detect reactions, which occurs near the transistor since the reactions will change the electrical environment slightly. In order to use the SET it is important to have a control over other stray charges as present in a buffer or derived from static electricity, since these charges would otherwise influence the result of the measurement.

Applications

DNA Sequencing

The methods described above can be used for studying hybridization of one single stranded DNA molecule to another single stranded DNA molecule that has been fixed between two electrodes. An array of different permutations of the same length of target DNA fixed between the two electrodes is used as target sequence in a hybridization reaction. The sequence of a DNA molecule (unknown) sequence of the same length as the target sequence will be detected as a change in capacitance, tunnelling or single electron tunnelling. Thus the molecule on the DNA array that has generated the largest change in capacitance or tunnel characteristics will contain a target sequence with a 100% complementarism to that of the unknown sequence. In short, a target sequence with a perfect match to that of the unknown sequence will generate the largest change in tunnelling and/or capacitance. Thus the present invention is used as a previously unknown way of sequencing DNA.

Protein Detection

Furthermore, any biomolecule with affinity for single or double stranded DNA, fixed between the two electrodes that alters the capacitance and/or tunnelling can be detected at low molecular concentrations.

Selection of DNA Molecules with High Affinity to a Protein

A protein that is allowed to bind to an array of DNA molecules, single or double stranded, will bind with different affinities to the various DNA sequences present. The binding reaction with the highest affinity will be detected as the largest change in capacitance and/or tunnelling.

Chemical Reaction Studies

The SET can be used to study the reaction rate or other characterization of a biochemical reaction or any other chemical reaction in the vicinity of the device. Thus any chemical reaction between a target molecule and an assay molecule will be monitored.

The term DNA molecule used herein is not restricted to single or double stranded DNA as such but relates to RNA=s, haptens, peptides, amino acids, DNA binding proteins, histones, polymerases, and ligases, and all other molecules, as well.

Experimental

In order to detect the impedance change due to the binding of assay target molecules to target molecules attached to a set of electrodes AC measurements were conducted using a Rodhe & Schwartz network analyzer in the range 20 kHz-8 GHz. A chip with the electrode configuration seen below in FIG. 5 was mounted in a metal measurement cell and connected to the network analyzer via SMA contacts, i.e. contacts specified for high frequencies.

The chip was fabricated by photolithography on a SiO₂ substrate. Gold (on top of titanium) electrodes were evaporated and lift-off was performed in acetone. The measurement cell was equipped with a flow system for adding and removing liquid to the inner electrodes of the chip.

The signal measured was S₂₁, i.e. how much of the input signal that goes through the device, in decibel.
S₂₁=10 log(Pₒᵤₜ/Pₐᵥ)=10 log(2Zᵢₙ/Zₜₒₜ)ˆ2
where Pₐᵥ is the available power applied, Pₒᵤₜ is the power over the device, Zᵢₙ is the 50 Ω resistance of the connecting cables, Zₜₒₜ is the total impedance of the device and the cables.

The largest shift of the S₂₁ signal would stem from the salt concentration of the buffer since the salt ions will work as charge carriers in the system. A typical salt dependence of the S₂₁ signal can be seen in FIG. 6 below. In order to detect the shift in S₂₁ signal as a result of the binding of assay target molecules to target molecules the salt concentration of the buffer was therefore kept constant through the whole experiment. After addition of any molecule the container was always rinsed with buffer so that comparison of the shift in S₂₁ could easily be done after different steps.

Two types of experiments were performed and these indicates that it is possible to detect small amounts of the protein avidin as well as small amounts of avidin coated gold particles is possible.

The protein coated gold particle were prepared by first boiling of HauCl₄ and Na₃ citrate in MilliQ-water to make the gold particles (different amount of Na₃ citrate gives rise to different sizes of particles) and secondly by adding avidin, 5000 avidin molecules per gold particle. The excess of avidin was removed from the solution by centrifugation. During centrifugation the avidin coated gold particles will form a pellet at the bottom of the test tube and the excess avidin in the solution can be removed from the gold particles, which are then diluted in 5 mM CaCl₂.

Before measuring the gold electrodes were coated by a self-assembled monolayer of alkanethiols. The chip was then rinsed with hexane and mounted in the measurements cell. TRIS buffer (10 mM TRIS, 5 mM CaCl₂, pH 8, Ca²⁺ prevents avidin from binding un-specifically to lipids) was added to the teflon container and 30 μl lipid liposomes were added in 1 ml buffer. Lipid liposomes are known to form bi-layer on SiO₂ and monolayer on thiols.(Reference: C. A. Keller, K. Glasmastar, V. P. Zhdanov and B. Kasemo, Physical Review Letters, 84, 23, (2000)). The lipid liposomes contained 5% of biotin labeled lipids and the biotin is the target molecule in this system. After bi- and monolayer formation 100 μl avidin (1 mg/ml) or avidin coated gold particles were added. Avidin, which is the assay target molecule in this model experiment, has four binding sites for biotin. The aim was to detect the binding between the biotin labeled lipids and the added avidin/avidin coated gold particles. The detection was made by studying the signal shift, i.e. the decrease of S₂₁ at different frequencies. S₂₁ as a function of frequency after the different steps can be seen in FIG. 7 below. The largest decrease, at 20 kHz, in the S₂₁-signal for avidin addition detected was more than 1.3 dB, which is a rather large and very detectable signal change. The method is sensitive enough to detect different amounts of avidin. The amounts have to be calibrated with complementary methods and this work is in progress. Addition of albumin, a protein which does not bind specifically to biotin, was also tested and then no decrease in the S₂₁ signal could be detected.

The result for the addition of avidin coated gold particles can be seen in FIG. 8. The largest decrease in the S₂₁-signal for avidin coated gold particles was more than 1 dB but less than for avidin at 20 kHz. This could be expected since the size of the gold particles was about 50 nm and therefore covered many of the biotin labeled lipids and therefore less avidin could bind. We believe that the decrease in S₂₁ is mainly due to the change in dielectric constant between buffer, εᵣ≈80, and the biomolecules, εᵣ≈3, since a linear slope in the S₂₁ curve indicates a capacitance. In order to be able to detect smaller amount of avidin it is important to minimize the area of gold electrodes exposed to the liquid. We are now working on miniaturization of the chip electrodes, to make it sensitive down to single molecular level. This is done by making the electrode gap smaller (down to 25 nm) and by covering most of the electrode by a thick layer of insulator, i.e. silicon dioxide, so that the important region is the gap between the electrodes. We also intend to develop a technique to cover the different electrodes in an array with different target molecules.

### FIGURE LEGENDS

FIG. 1. Detection of target biomolecule adsorption by AC conductance. measurement

FIG. 2. Charging and discharging of a capacitor

FIG. 3. A schematic picture of a SET transistor

FIG. 4. The charging energy of a capacitance as a function of the charge

FIG. 5. Electrode configuration on chip, distance between electrodes are 10, 20, 30, 40 resp. 50 micron.

FIG. 6. Test of the chip sensitivity to different concentrations of NaCl in 10 mM TRIS buffer: a) 100 mM NaCl, b) 50 mM NaCl, c) 10 mM NaCl, d) 5 mM NaCl, a) 0 mM NaCl and f)air.

FIG. 7. S₂₁ for a) thiol covered electrodes in buffer, b) after bi-layer formation, c) after binding of avidin and d) electrodes in air.

FIG. 8. S₂₁ at 20 kHz for 1) electrodes in buffer, 2) electrodes covered by thiols, 3) bi-layer between electrodes and monolayer of lipids (5% biotin labeled lipids) on top of thiols and 4) Avidin coated gold particles binding to biotin in lipid bi- and monolayer

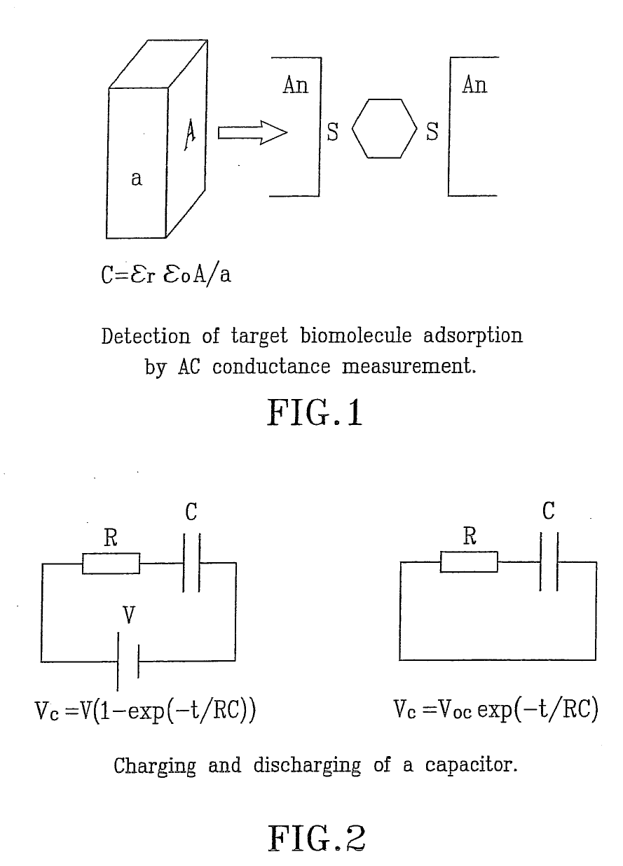

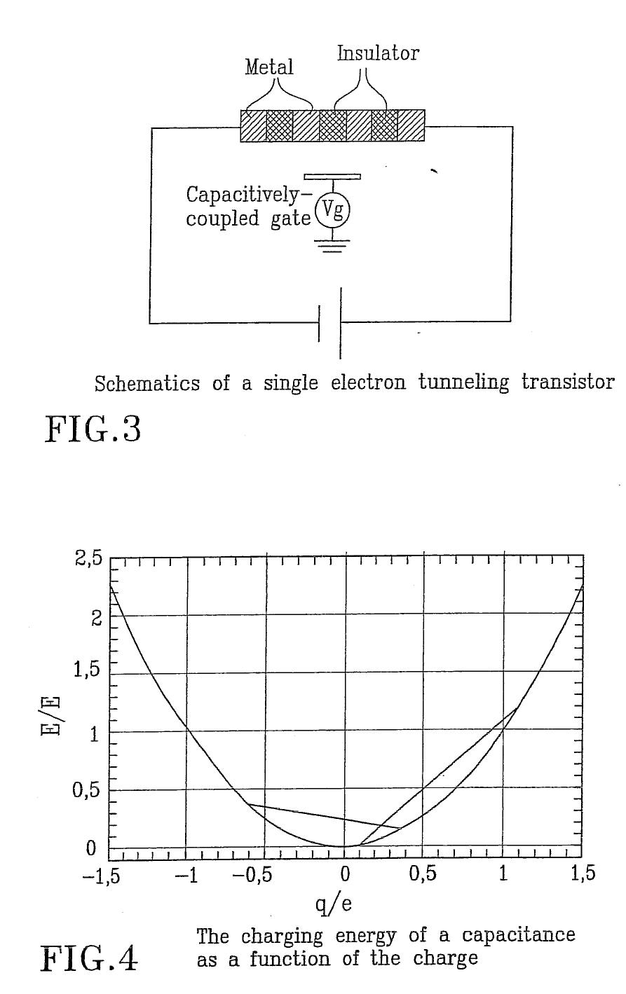

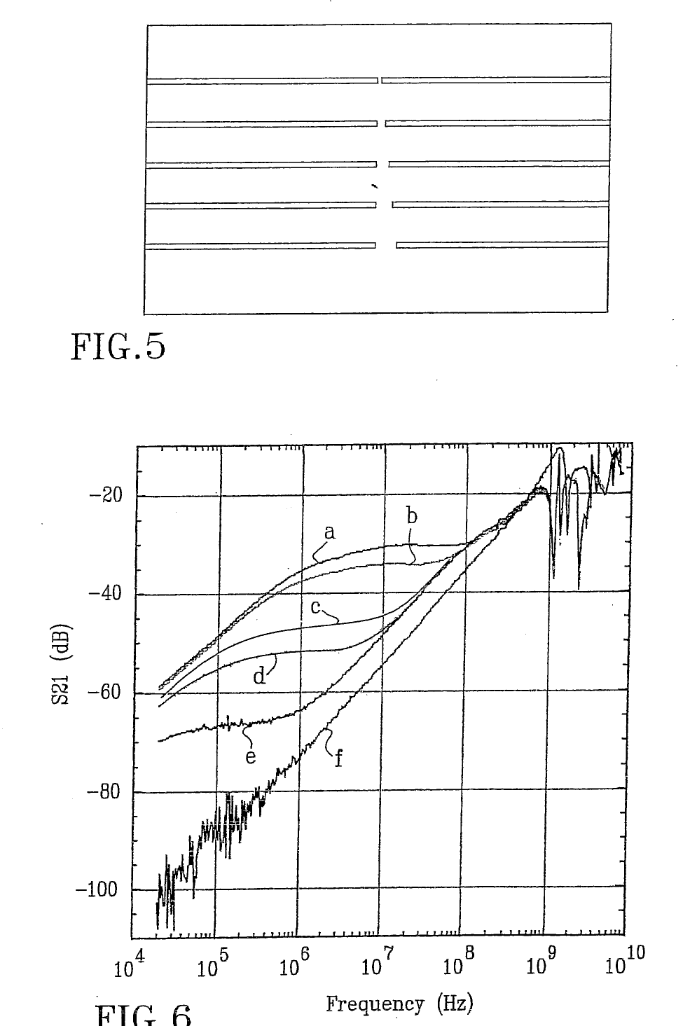

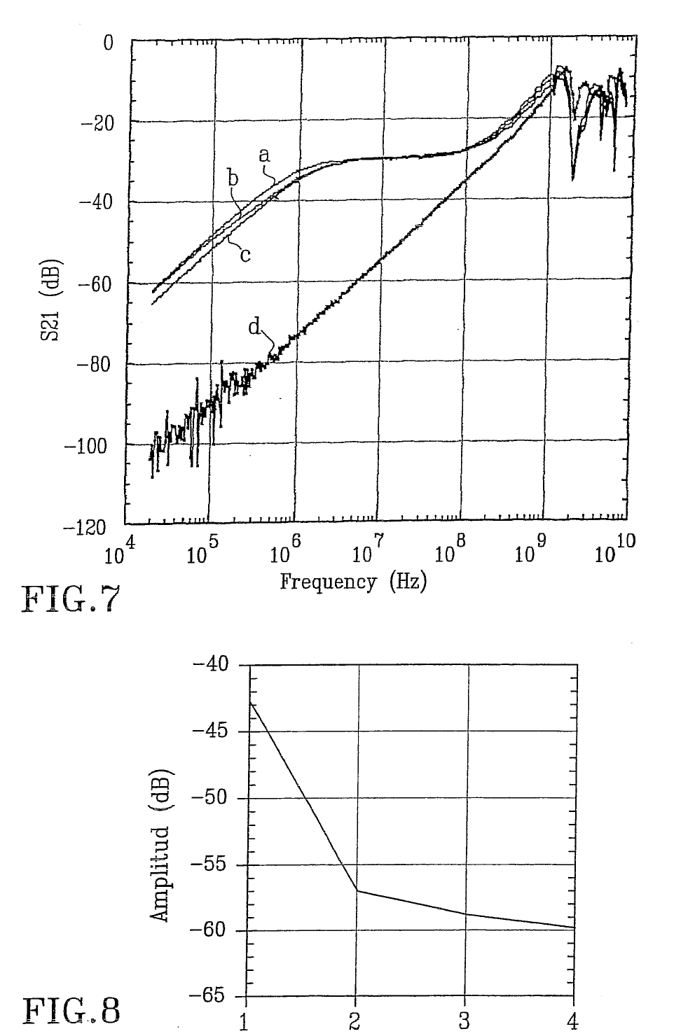

### REFERENCES

1. A self-assembled single-electron tunnelling transistor, S. H. M. Persson, L. Olofsson, L. Gunnarsson, Appl. Phys. Lett. Vol 74; no. 17; p 2546-8 (1999)

2. Nanofabrication of self-assembled molecular-scale electronics Linda G. M. Olofsson, S. H. Magnus Persson, Alberto Morpurgo, Charles M. Marcus, Dimitri Golubev, Linda K. Gunnarsson, and Yimin Yao, J. Low Temp. Phys., vol 118, no. 5, p 343,(2000)

3. Principles of electron tunnelling spectroscopy E. L. Wolf, Oxford University Press (1985)

4. D. Porath, A. Bezryadin, S. de Vries, and C. Dekker, preprint (1999)

5. H. W. Fink C. Schonenberger, Nature, vol 398, no. 6726, p. 407, (1999)

6. K. K. Likharev, IEEE Transactions on magnetics 23, vol. 2; p. 1142 (1987)

7. D. V. Averin, K. K. Likharev, Single electronics: A correlated transfer of single electrons and Cooper pairs in systems of small tunnel junctions. In Mesoscopic Phenomena in Solids.

8. Single electron tunnelling, L. I. Glazman, J. Low Temp. Phys. vol 118, no. 5, p 247, (2000)

1. A detection method for detecting molecules and/or chemical reactions, wherein target molecules are attached to a series of electrodes, the series of electrodes are subjected to assay target molecules or other molecules of interest, whereupon detection of signal losses, detection of dielectricum changes and/or detection of electron charge changes detection is determined by means of blockade.

2-5. (canceled)

6. The method according to claim 1, wherein the molecules are organic chemical molecules.

7. The method according to claim 1, wherein the molecules are biomolecules.

8. The method according to claim 1, wherein the molecules are inorganic chemical molecules.

9. The method according to claim 1, wherein the molecules to be detected are attached to a substrate having no conductive top layer.

10. The method according to claim 9, wherein the top layer is of silicon.

11. The method according to claim 9, wherein the top layer is of glass.

12. The method according to claim 1, wherein the electrodes are present as elevated dots on a chip.

13. A detection method for detecting biomolecules and/or biochemical reactions, wherein target molecules are attached to a series of electrodes, the series of electrodes are subjected to assay target molecules or other molecules of interest, whereupon detection of signal losses, detection of dielectricum changes and/or detection of electron charge changes detection is determined by means of Coulumb blockade.
